(19) United States
(12) Patent Application Publication
(10) Pub. No.: US 2024/0013866 A1
(43) Pub. Date: Jan. 11, 2024

(54) MACHINE LEARNING FOR PREDICTING THE PROPERTIES OF CHEMICAL FORMULATIONS

(71) Applicant: Osmo Labs, PBC, Cambridge, MA (US)

(72) Inventors: Brian Kihoon Lee, Somerville, MA (US); Alexander Wiltschko, Somerville, MA (US)

(21) Appl. No.: 18/370,711

(22) Filed: Sep. 20, 2023

Related U.S. Application Data
(63) Continuation of application No. PCT/US21/63436, filed on Dec. 15, 2021.
(60) Provisional application No. 63/165,781, filed on Mar. 25, 2021.

(51) Int. Cl. G16C 20/30; G16C 20/70

(52) U.S. Cl. CPC G16C 20/30; G16C 20/70

(57) ABSTRACT

Chemical formulation property prediction can involve understanding each molecule individually and the mixture as a whole. Machine-learned models can be utilized to extract individual and holistic data to generate accurate predictions of the properties of mixtures. Properties that can include, but are not limited to, olfactory properties, taste properties, color properties, viscosity properties, and other commercially, industrially, or pharmaceutically beneficial properties.

### RELATED APPLICATIONS

This application claims priority to and the benefit of U.S. Provisional Patent Application No. 63/165,781, filed Mar. 25, 2021. U.S. Provisional Patent Application No. 63/165,781 is hereby incorporated by reference in its entirety.

### FIELD

The present disclosure relates generally to predicting the properties of chemical formulations using machine learning. More particularly, the present disclosure relates to property prediction using properties of molecules, concentrations, composition, and interactions.

### BACKGROUND

The vast majority of chemical products are not single molecules, but carefully crafted formulations or mixtures. The field of machine learning for chemistry has advanced rapidly in being able to predict the physical and perceptual properties of single, isolated molecules, but chemical formulations are largely ignored.

Mixture models in the art focus on perceptual similarity of mixtures for predictions while ignoring other factors. For example, certain existing approaches focus on storing and providing human acquired data on properties of mixtures such as human tasted mixtures. The stored data relies on human acquired data, which can lead to subjective bias, including varying scales based on the acquirer of data.

### SUMMARY

Aspects and advantages of embodiments of the present disclosure will be set forth in part in the following description, or can be learned from the description, or can be learned through practice of the embodiments.

One example aspect of the present disclosure is directed to a computer-implemented method for mixture property prediction. The method can include obtaining, by a computing system comprising one or more computing devices, respective molecule data for each of a plurality of molecules and mixture data associated with a mixture of the plurality of molecules. The method can include respectively processing, by the computing device, the respective molecule data for each of the plurality of molecules with a machine-learned embedding model to generate a respective embedding for each molecule. The method can include processing, by the computing system, the embeddings and the mixture data with a prediction model to generate one or more property predictions for the mixture of the plurality of molecules. In some implementations, the one or more property predictions can be based at least in part on the embeddings and the mixture data. The method can include storing, by the computing system, the one or more property predictions.

In some implementations, the mixture data can describe a respective concentration of each molecule in the mixture. The mixture data can describe a composition of the mixture. The prediction model can include a deep neural network. In some implementations, the machine-learned embedding model can include a machine-learned graph neural network. The prediction model can include a characteristic-specific model configured to generate predictions relative to a specific characteristic. The one or more property predictions can be based at least in part on a binding energy of one or more molecules of the plurality of molecules. In some implementations, the one or more property predictions can include one or more sensory property predictions. The one or more property predictions can include an olfactory prediction. The one or more property predictions can include a catalytic property prediction. In some implementations, the one or more property predictions can include an energetic property prediction. The one or more property predictions can include a surfactant between target property prediction.

In some implementations, the one or more property predictions can include a pharmaceutical property prediction. The one or more property predictions can include a thermal property prediction. The prediction model can include a weighting model configured to weight and pool the embeddings based on the mixture data, and the mixture data can include concentration data related to the plurality of molecules of the mixture.

In some implementations, the method can include obtaining, by the computing system, a request from a requesting computing device for a chemical mixture with a requested property, determining, by the computing system, the one or more property predictions satisfy the requested property, and providing, by the computing system, the mixture data to the requesting computing device. The one or more property predictions can be based at least in part on a molecule interaction property. In some implementations, the one or more property predictions can be based at least in part on receptor activation data.

Another example aspect of the present disclosure is directed to a computing system. The computing system can include one or more processors and one or more non-transitory computer readable media that collectively store instructions that, when executed by the one or more processors, cause the computing system to perform operations. The operations can include obtaining respective molecule data for a plurality of molecules and mixture data associated with a mixture of the plurality of molecules. In some implementations, the mixture data can include concentrations for each respective molecule of the plurality of the molecules. The operations can include respectively processing the respective molecule data with an embedding model for each of the plurality of molecules to generate respective embeddings for each molecule. The operations can include processing the embeddings and the mixture data with a machine-learned prediction model to generate one or more property predictions. The one or more property predictions can be based at least in part on the embeddings and the mixture data. The operations can include storing the one or more property predictions.

Another example aspect of the present disclosure is directed to one or more non-transitory computer readable media that collectively store instructions that, when executed by one or more processors, cause a computing system to perform operations. The operations can include obtaining respective molecule data for a plurality of molecules and mixture data associated with a mixture of the plurality of molecules. The operations can include respectively processing the respective molecule data with an embedding model for each of the plurality of molecules to generate respective embeddings for each molecule. The operations can include processing the embeddings and the mixture data with a machine-learned prediction model to generate one or more property predictions. In some implementations, the one or more property predictions can be based at least in part on the embeddings and the mixture data. The operations can include storing the one or more property predictions.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1A depicts a block diagram of an example computing system that performs mixture property prediction according to example embodiments of the present disclosure.

FIG. 1B depicts a block diagram of an example computing device that performs mixture property prediction according to example embodiments of the present disclosure.

FIG. 1C depicts a block diagram of an example computing device that performs mixture property prediction according to example embodiments of the present disclosure.

FIG. 2 depicts a block diagram of an example machine-learned prediction model according to example embodiments of the present disclosure.

FIG. 3 depicts a block diagram of an example property prediction model system according to example embodiments of the present disclosure.

FIG. 4 depicts a block diagram of an example property request system according to example embodiments of the present disclosure.

FIG. 5 depicts a block diagram of an example mixture property profile according to example embodiments of the present disclosure.

FIG. 6 depicts a flow chart diagram of an example method to perform mixture property prediction according to example embodiments of the present disclosure.

FIG. 7 depicts a flow chart diagram of an example method to perform property prediction and retrieval according to example embodiments of the present disclosure.

FIG. 8 depicts a flow chart diagram of an example method to perform property prediction database generation according to example embodiments of the present disclosure.

FIG. 9A depicts a block diagram of an example evolutionary approach according to example embodiments of the present disclosure.

FIG. 9B depicts a block diagram of an example reinforcement learning approach according to example embodiments of the present disclosure.

Reference numerals that are repeated across plural figures are intended to identify the same features in various implementations.

### DETAILED DESCRIPTION

### Overview

Generally, the present disclosure is directed to systems and methods for using machine learning to predict one or more properties of a mixture of multiple chemical molecules. The systems and methods can leverage known properties for individual molecules, compositions, and interactions to predict properties for mixtures before the mixture is tested. Moreover, machine-learned models can be used to utilize artificial intelligence techniques to quickly and efficiently predict the properties of the mixtures. The systems and methods can include obtaining molecule data for one or more molecules and mixture data associated with a mixture of the one or more molecules. The molecule data can include respective molecule data for each molecule of a plurality of molecules that make up a mixture. In some implementations, the mixture data can include data related to the concentration of each molecule in the mixture along with the overall composition of the mixture. The mixture data can describe the chemical formulation of the mixture. The molecule data can be processed with an embedding model to generate a plurality of embeddings. Each respective molecule data for each respective molecule may be processed with the embedding model to generate a respective embedding for each respective molecule in the mixture. In some implementations, the embeddings can include data descriptive of individual molecule properties for the embedded data. In some implementations, the embeddings can be vectors of numbers. In some cases, the embeddings may represent graphs or molecular property descriptions. The embeddings and the mixture data can be processed by a prediction model to generate one or more property predictions. The one or more property predictions can be based at least in part on the one or more embeddings and the mixture data. The property predictions can include various predictions on the taste, smell, coloration, etc. of the mixture. In some implementations, the systems and methods can include storing the one or more property predictions. In some implementations, one or both of the models can include a machine-learned model.

Obtaining molecule data and mixture data can include receiving a request for property predictions for a mixture including the one or more molecules of the plurality of molecules. The request can further include concentrations for each of the one or more molecules. The request can include characteristic specific properties (e.g., sensory properties) or mixture properties in general. Alternatively or additionally, obtaining molecule data and mixture data can include a form of sampling, such as random sampling or category specific sampling. For example, random sampling of molecule mixtures may be implemented to catalog predictions of various mixtures. Alternatively, category specific sampling can include taking molecules in a category with known properties and sampling with molecules in another category with other known properties.

After the molecule data is obtained, the molecule data can be processed with an embedding model to generate a plurality of embeddings. Each molecule of the plurality of molecules may receive one or more respective embeddings. The embeddings may be property feature embeddings, which can include embedded data related to individual molecule properties. For example, an embedding for a first molecule may include embedded information descriptive of the olfactory properties of that molecule. In some implementations, the embedding model can include a graph neural network that generates one or more embeddings for each respective molecule. In some implementations, the embeddings can be vectors, and the vectors can be based on processed graphs, in which the graphs describe one or more molecules.

The one or more embeddings can be processed with the mixture data by a prediction model to generate one or more property predictions. The prediction model can include weighting the one or more embeddings based on the concentration of the molecule the embedding is associated with. For example, a mixture including a first molecule and a second molecule with a two to one concentration ratio may include a heavier weighting for the embedding of the first molecule as the first molecule has a higher concentration in the mixture. Moreover, the machine-learned prediction model can include a weighting model including weighting and pooling the embeddings based on the mixture data, in which the mixture data can include concentration data related to the plurality of molecules of the mixture.

In some implementations, the prediction model can be a machine-learned prediction model, and the machine-learned prediction model can include a characteristic specific model (e.g., sensory property prediction model, energetic property prediction model, thermal property prediction model, etc.).

After being generated, the one or more property predictions can be stored. The predictions can be stored in a database of property predictions and may be stored on a centralized server. In some implementations, the predictions may be provided to a computing device after being generated. The stored predictions may be organized into a mixture property prediction profile, which can include the mixture and its respective property predictions in a digestible format.

The stored predictions may be received upon request. In some implementations, the stored predictions can be readily searchable. For example, the system can receive a request for a particular property in the form of a property search query. The system can determine if the requested property is one of the properties in the property predictions for the mixture. If the requested property is in the property predictions, the mixture information may be provided to the requestor.

In some implementations, property predictions can be based on one or more initial predictions, including, but not limited to: predicting a single molecule's properties as a function of concentration, predicting a mixture's properties as a function of mixture composition, and predicting a mixture's properties when components of the mixture interact (e.g., synergistically or competitively). Each prediction may be generated by a separate model or by a singular model. The systems and methods may rely on an algorithm that is fully differentiable. In some implementations, the systems and methods may use knowledge of strong chemical inductive biases and nonconvex optimization for training their predictive models. Furthermore, the machine-learned models can be trained using gradient descent and a dataset of mixture data. In some implementations, the machine-learned prediction model may be trained with a training dataset with labeled pairings. In some implementations, the training data can include known receptor activation data.

In some implementations, the systems and methods can predict the perceptual or physical properties of mixtures. The methods and systems can involve explicitly modeling chemically realistic equilibrium and competitive binding dynamics, where the entire algorithm can be fully differentiable. This implementation can allow both the use of strong chemical inductive biases, and also the full toolkit of nonconvex optimization from the field of neural networks and machine learning.

More specifically, the machine-learned prediction model can be trained for concentration dependence and modeling mixtures, which can include mixtures with competitive inhibition and mixtures with noncompetitive inhibition. Concentration dependence can include understanding the properties of individual molecules and factoring in and weighting the properties of individual molecules based on the concentration of each molecule in the mixture.

Mixtures with competitive inhibition can include mixtures in which the various molecules of the mixture are competing for activating a receptor (e.g., molecules competing to activate an odor receptor). Moreover, the systems and methods can factor in that molecules with higher normalized binding energy can be more likely to trigger receptors before lower normalized binding energy molecules. In some implementations, the mixtures with competitive inhibition can be considered by the system by adding a second head to the model. One head can model the net binding energy, the other head can model the “proper substrate or competitive inhibitor” propensity score, and the two heads can be elementwise multiplied. The systems and methods can include an attention mechanism. The two headed model can factor in which molecule activates a receptor.

Mixtures with noncompetitive inhibition can include cumulative inhibition based on a proper activation binding mode and a noncompetitively inhibiting binding mode.

In some implementations, the weighting of the embeddings based on concentration can be a weighted average. The weighting can generate a single fixed-dimensional embedding. In some implementations, the concentration can be passed through a nonlinearity. In some implementations, a weighting model can generate a weighted set of graphs. Moreover, in some implementations, the graph structures of the molecules in a mixture may be passed in as a weighted set to a neural network model, and a machine learning method to handle variable-sized set input may be used to digest each molecule. For instance, methods such as set2vec may be combined with graph neural network methods.

Furthermore, the graph structures of the molecules in a mixture may be embedded in a “graph of graphs,” where each node represents a molecule in the mixture. The edges may be constructed in an all-to-all fashion (e.g., hypothesizing that all molecule types may interact with each other) or using chemical prior knowledge to prune down the interactions between molecules that are more or less likely to occur. In some implementations, the edges may be weighted according to the likelihood of interaction. Then, standard graph neural network methods may be used to pass messages both within the atoms of molecules, and between entire molecules, in an alternating fashion.

In some implementations, the systems and methods can include a nearest neighbor interpolation. A nearest neighbor interpolation can include enumerating a set of N ingredients and can include representing each mixture as an N-dimensional vector. The vector can represent the proportion of each ingredient. A prediction for a novel mixture can involve a nearest-neighbor lookup according to some distance metric, followed by an averaging of the perceptual properties for the nearest neighbors. The averaged perceptual properties can be the predictions.

Alternatively or additionally, in some implementations, the systems and methods can include direct molecular dynamics simulation, through a quantum mechanics based or molecular force field based approach. For example, each molecule's interaction with a putative odor receptor or taste receptor can be directly modeled using specialized computers for molecular simulation, and the strength of the interaction can be measured by the simulation. The perceptual properties of a mixture may be modeled based on the combined interactions of all components.

The property predictions can include sensory property predictions (e.g., olfactory properties, taste properties, color properties, etc.). Additionally and/or alternatively, the property predictions can include catalytic property predictions, energetic property predictions, surfactant between target property predictions, pharmaceutical property predictions, odor quality predictions, odor intensity predictions, color predictions, viscosity predictions, lubricant property predictions, boiling point predictions, adhesion property predictions, coloration property predictions, stability predictions, and thermal property predictions. For example, the property predictions can include predictions related to properties that can be beneficial to battery design, such as how long the mixture holds a charge, how much charge the mixture can hold, discharge, rate, degradation rate, stability, and overall quality.

The systems and methods disclosed herein can be applied to generate property predictions for a variety of uses including but not limited to consumer packaged goods, flavor and fragrance, and industrial applications such as dyes, paints, lubricants, and energy applications such as battery design.

In some embodiments, the systems and methods described herein can be implemented by one or more computing devices. the computing device(a) can include one or more processors and one or more non-transitory computer-readable media that store instructions that, when executed by the one or more processors, cause the computing device to perform operations. The operations can include steps of various methods described herein.

In some implementations, the systems and methods disclosed herein can be used for a closed-loop development process. For example, a human practitioner can utilize the systems and methods disclosed herein to predict the properties of mixtures before physically creating the mixture. In some implementations, the systems and methods can be used to generate a database of theoretical mixtures with predicted properties. A human practitioner can utilize the generated database to enable computer-aided mixture design for a desired effect. Moreover, the database may be a searchable database that can be used to screen through all possible mixtures to identify mixtures with desired perceptual and physical properties.

For example, a human practitioner may be attempting to make a new, potent flowery fragrance. The human practitioner may provide theoretical mixture suggestions to the embedding model and machine-learned prediction model to output predicted properties of the theoretical mixtures. The human practitioner can use the predictions to determine whether to actually produce the mixture or continue formulating other mixtures for testing. In some implementations, in response to determining one or more mixtures are predicted to have the desired properties, the system may send instructions to a manufacturing system or a user computing system to manufacture the one or more mixtures for physical testing.

Alternatively and/or additionally, the human practitioner may search or screen through mixtures that have already been processed by the machine-learned model(s) to generate property predictions. The mixtures and their respective property predictions can be stored in a database to provide ease in screening through or searching the data. A human practitioner can screen through the plurality of mixtures to find mixtures with property predictions that match a desired property. For example, the human practitioner attempting to make a new, potent flowery fragrance may screen through the database for a mixture predicted to have a potent smell with flowery notes.

The closed-loop development process utilization of the systems and methods disclosed herein can save time and can save on cost of producing and physically testing mixtures. Human practitioners can screen through data with the machine-learned models to quickly eliminate a large amount of possible mixtures from the pool of possible candidates. Moreover, the machine-learned models may predict properties that indicate candidate mixtures that may be overlooked by human practitioners due to the candidate mixtures having surprise cumulative properties.

In some implementations, the systems and methods for using machine learning to predict one or more properties of a mixture of multiple chemical molecules may be used to control machinery and/or provide an alert. The systems and methods can be used to control manufacturing machinery to provide a safer work environment or to change the composition of a mixture to provide a desired output. Moreover, in some implementations, the property prediction can be processed to determine if an alert needs to be provided. For example, in some implementations, the property predictions may include olfactory property predictions for the scent of a vehicle used for transportation services. The systems and methods may output scent profile predictions, potency predictions, and scent lifetime predictions for an air freshener, a fragrance, or a candle alternative. The predictions can then be processed to determine when a new product should be placed in the transportation device and/or whether the transportation device should undergo a cleaning routine. The determined new product time may then be sent as an alert to a user computing device or may be used to set up an automated purchase. In another example, the transportation device (e.g., an autonomous vehicle) may be automatically recalled to a facility to undergo a cleaning routine. In another example, an alert can be provided in a property prediction generated by the machine learning model indicates an unsafe environment for animals or persons present within a space. For example, an audio alert can sound in a building if a prediction of lack of safety is generated for a mixture of chemical molecules sensed to be in the building.

In some implementations, the system may intake sensor data to be input into the embedding model and prediction model to generate property predictions of the environment. For example, the system may utilize one or more sensors for intaking data associated with the presence and/or concentration of molecules in the environment. The system can process the sensor data to generate input data for the embedding model and the prediction model to generate property predictions for the environment, which can include one or more predictions on the smell of the environment or other properties of the environment. If the predictions include a determined unpleasant odor, the system may send an alert to a user computing device to have a cleaning service completed. In some implementations, the system may bypass an alert and send an appointment request to a cleaning service upon determination of the unpleasant odor.

Another example implementation can involve background processing and/or active monitoring for safety precautions. For example, the system can document manufacturing steps completed by a user or a machine to track the predicted property of created mixtures to ensure the manufacturer is aware of any dangers. In some implementations, upon selection of a new molecule or mixture being added to the ongoing mixture, the new potential mixture may be processed by the embedding model and prediction model to determine the property predictions of the new mixture. The property predictions can include whether the new mixture is flammable, poisonous, unstable, or dangerous in any way. If the new mixture is determined to be dangerous in any way, an alert may be sent. Alternatively and/or additionally, the system may control one or more machines to stop and/or contain the process to protect from any potential present or future danger.

The systems and methods can be applied to other manufacturing, industrial, or commercial systems to provide automated alerts or automated actions in response to property predictions. These applications can include new mixture creations, adjustments to recipes, counteracting measures, or real-time alerts on changes in predicted properties.

The systems and methods of the present disclosure provide a number of technical effects and benefits. As one example, the system and methods can provide property predictions for mixtures without having to individually and physically test various mixtures of molecules. The systems and methods can further be used to generate a database of mixtures with predicted properties that can be readily searchable for finding mixtures with certain properties to be implemented in fragrances, foods, lubricants, and so forth based on their predicted properties. Furthermore, the systems and methods can enable more accurate predictions due to consideration of both individual molecule properties and interaction properties. Thus, the ability of a computer to perform a task (e.g., a mixture fragrance prediction) can be improved.

Another technical benefit of the systems and methods of the present disclosure is the ability to quickly and efficiently predict mixture properties, which can circumvent the need for testing mixtures with human taste tests and other physical testing applications.

With reference now to the Figures, example embodiments of the present disclosure will be discussed in further detail.

### Example Devices and Systems

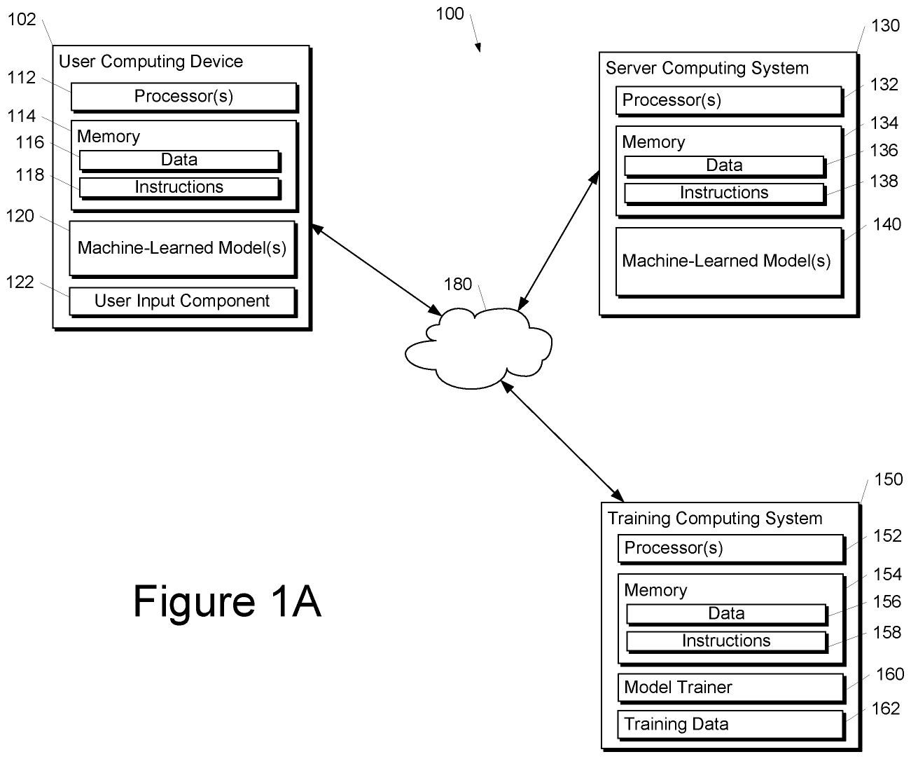

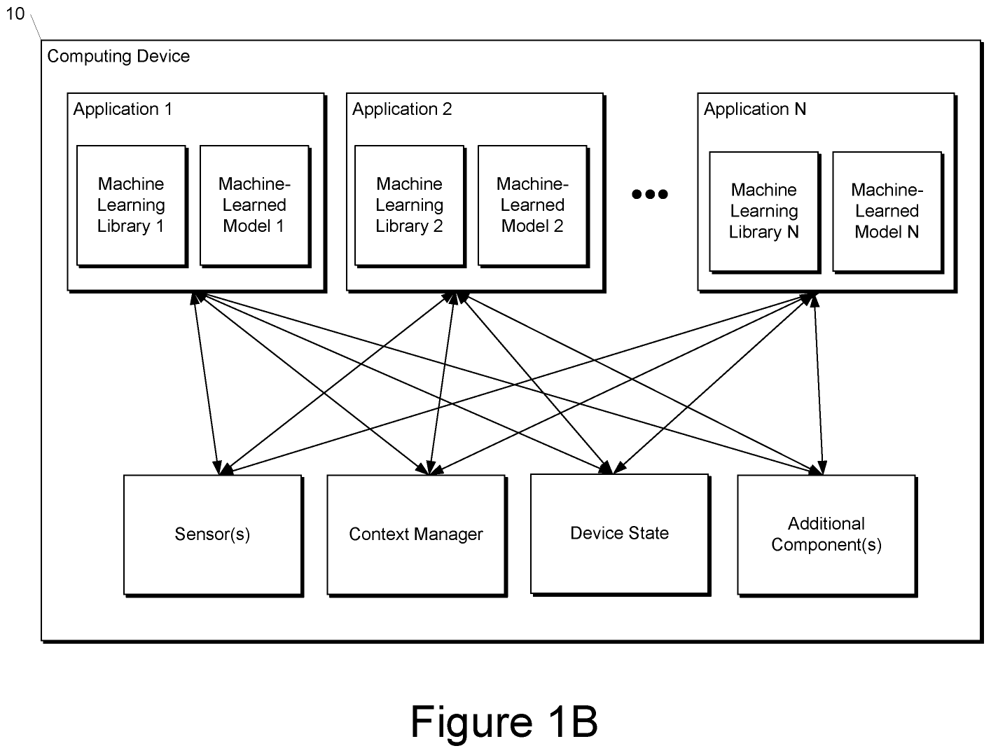

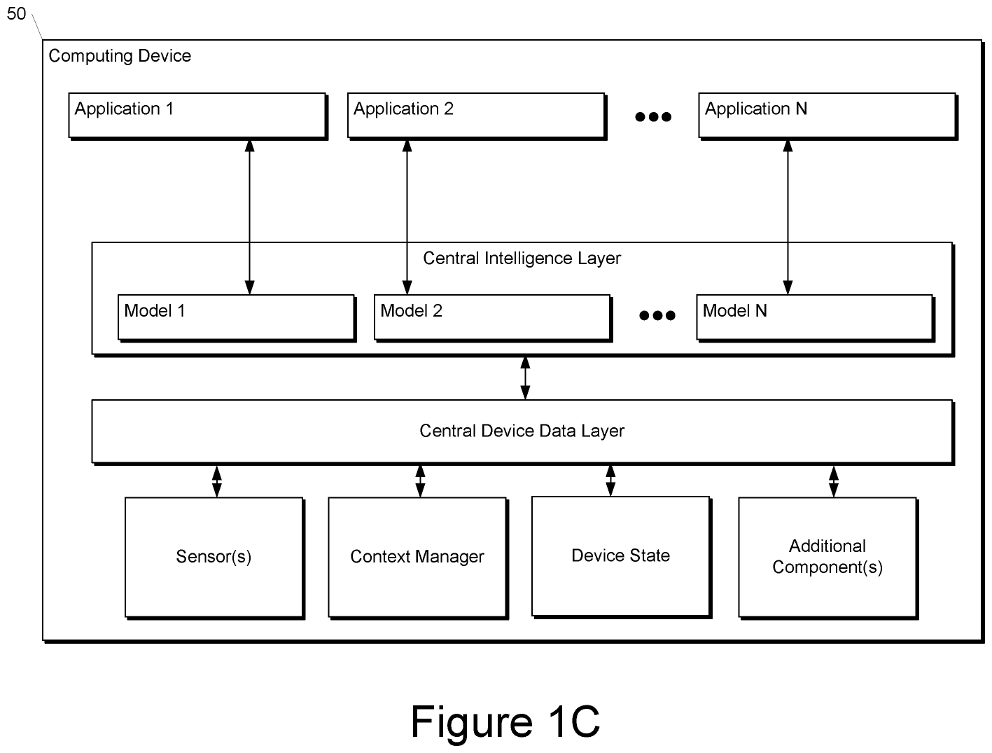

FIG. 1A depicts a block diagram of an example computing system 100 that performs property predictions according to example embodiments of the present disclosure. The system 100 includes a user computing device 102, a server computing system 130, and a training computing system 150 that are communicatively coupled over a network 180.

The user computing device 102 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 102 includes one or more processors 112 and a memory 114. The one or more processors 112 can be any suitable processing device (e.g., a processor core, a microprocessor, an ASIC, a FPGA, a controller, a microcontroller, etc.) and can be one processor or a plurality of processors that are operatively connected. The memory 114 can include one or more non-transitory computer-readable storage mediums, such as RAM, ROM, EEPROM, EPROM, flash memory devices, magnetic disks, etc., and combinations thereof. The memory 114 can store data 116 and instructions 118 which are executed by the processor 112 to cause the user computing device 102 to perform operations.

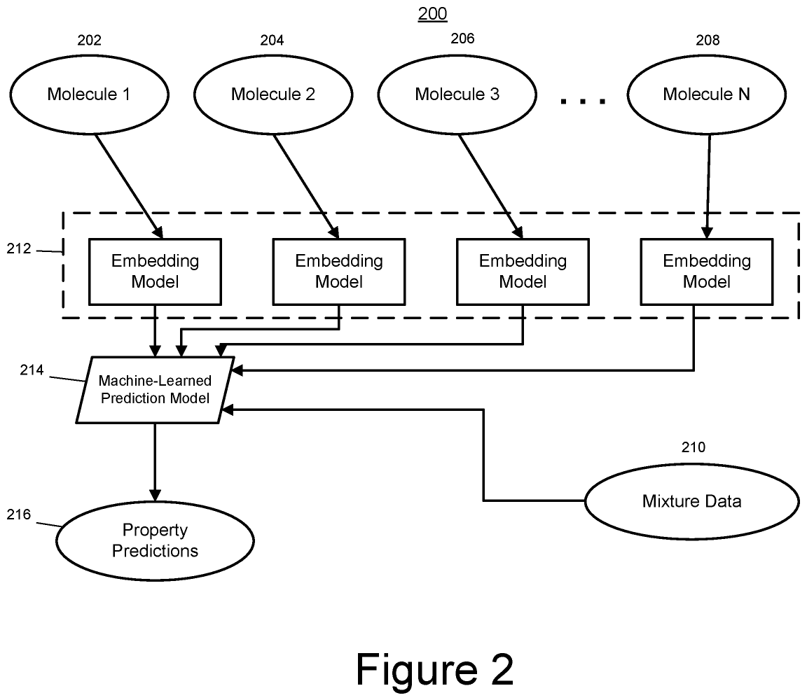

In some implementations, the user computing device 102 can store or include one or more prediction models 120. For example, the prediction models 120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. Example prediction models 120 are discussed with reference to FIGS. 2, 3, and 6-8.

In some implementations, the one or more prediction models 120 can be received from the server computing system 130 over network 180, stored in the user computing device memory 114, and then used or otherwise implemented by the one or more processors 112. In some implementations, the user computing device 102 can implement multiple parallel instances of a single prediction model 120 (e.g., to perform parallel mixture property predictions across multiple instances of mixture composition).

More particularly, the machine-learned prediction model can be trained to intake molecule data and mixture data and output property predictions for the mixture the mixture data is descriptive of In some implementations, the molecule data may be embedded with an embedding model before being processed by a prediction model.

Additionally or alternatively, one or more prediction models 140 can be included in or otherwise stored and implemented by the server computing system 130 that communicates with the user computing device 102 according to a client-server relationship. For example, the prediction models 140 can be implemented by the server computing system 140 as a portion of a web service (e.g., a mixture property prediction service). Thus, one or more models 120 can be stored and implemented at the user computing device 102 and/or one or more models 140 can be stored and implemented at the server computing system 130.

The user computing device 102 can also include one or more user input component 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 130 includes one or more processors 132 and a memory 134. The one or more processors 132 can be any suitable processing device (e.g., a processor core, a microprocessor, an ASIC, a FPGA, a controller, a microcontroller, etc.) and can be one processor or a plurality of processors that are operatively connected. The memory 134 can include one or more non-transitory computer-readable storage mediums, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

As described above, the server computing system 130 can store or otherwise include one or more machine-learned prediction models 140. For example, the models 140 can be or can otherwise include various machine-learned models. Example machine-learned models include neural networks or other multi-layer non-linear models. Example neural networks include feed forward neural networks, deep neural networks, recurrent neural networks, and convolutional neural networks. Example models 140 are discussed with reference to FIGS. 2, 3, and 6-8.

The user computing device 102 and/or the server computing system 130 can train the models 120 and/or 140 via interaction with the training computing system 150 that is communicatively coupled over the network 180. The training computing system 150 can be separate from the server computing system 130 or can be a portion of the server computing system 130.

The training computing system 150 includes one or more processors 152 and a memory 154. The one or more processors 152 can be any suitable processing device (e.g., a processor core, a microprocessor, an ASIC, a FPGA, a controller, a microcontroller, etc.) and can be one processor or a plurality of processors that are operatively connected. The memory 154 can include one or more non-transitory computer-readable storage mediums, such as RAM, ROM, EEPROM, EPROM, flash memory devices, magnetic disks, etc., and combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the machine-learned models 120 and/or 140 stored at the user computing device 102 and/or the server computing system 130 using various training or learning techniques, such as, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a number of training iterations.

In some implementations, performing backwards propagation of errors can include performing truncated backpropagation through time. The model trainer 160 can perform a number of generalization techniques (e.g., weight decays, dropouts, etc.) to improve the generalization capability of the models being trained.

In particular, the model trainer 160 can train the prediction models 120 and/or 140 based on a set of training data 162. The training data 162 can include, for example, labeled training data, such as molecule data with known molecule property labels, mixture data with known composition property labels, and mixture data with known interaction property labels.

In some implementations, if the user has provided consent, the training examples can be provided by the user computing device 102. Thus, in such implementations, the model 120 provided to the user computing device 102 can be trained by the training computing system 150 on user-specific data received from the user computing device 102. In some instances, this process can be referred to as personalizing the model.

The model trainer 160 includes computer logic utilized to provide desired functionality. The model trainer 160 can be implemented in hardware, firmware, and/or software controlling a general purpose processor. For example, in some implementations, the model trainer 160 includes program files stored on a storage device, loaded into a memory and executed by one or more processors. In other implementations, the model trainer 160 includes one or more sets of computer-executable instructions that are stored in a tangible computer-readable storage medium such as RAM hard disk or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

The machine-learned models described in this specification may be used in a variety of tasks, applications, and/or use cases.

In some implementations, the input to the machine-learned model(s) of the present disclosure can be image data. The machine-learned model(s) can process the image data to generate an output. As an example, the machine-learned model(s) can process the image data to generate an image recognition output (e.g., a recognition of the image data, a latent embedding of the image data, an encoded representation of the image data, a hash of the image data, etc.). As another example, the machine-learned model(s) can process the image data to generate a molecular graph output, which can then be processed by the embedding model and the prediction model to generate property predictions.

In some implementations, the input to the machine-learned model(s) of the present disclosure can be text or natural language data. The machine-learned model(s) can process the text or natural language data to generate an output. As an example, the machine-learned model(s) can process the natural language data to generate a search query output. The search query output can be processed by a search model to search for a mixture with a particular property and output one or mixtures with that specific property. As another example, the machine-learned model(s) can process the text or natural language data to generate a classification output. The classification output can be descriptive of a mixture having one or more predicted properties. As another example, the machine-learned model(s) can process the text or natural language data to generate a prediction output.

In some implementations, the input to the machine-learned model(s) of the present disclosure can be latent encoding data (e.g., a latent space representation of an input, etc.). The machine-learned model(s) can process the latent encoding data to generate an output. As an example, the machine-learned model(s) can process the latent encoding data to generate a recognition output. As another example, the machine-learned model(s) can process the latent encoding data to generate a reconstruction output. As another example, the machine-learned model(s) can process the latent encoding data to generate a search output. As another example, the machine-learned model(s) can process the latent encoding data to generate a reclustering output. As another example, the machine-learned model(s) can process the latent encoding data to generate a prediction output.

In some implementations, the input to the machine-learned model(s) of the present disclosure can be statistical data. The machine-learned model(s) can process the statistical data to generate an output. As an example, the machine-learned model(s) can process the statistical data to generate a recognition output. As another example, the machine-learned model(s) can process the statistical data to generate a prediction output. As another example, the machine-learned model(s) can process the statistical data to generate a classification output. As another example, the machine-learned model(s) can process the statistical data to generate a segmentation output. As another example, the machine-learned model(s) can process the statistical data to generate a segmentation output. As another example, the machine-learned model(s) can process the statistical data to generate a visualization output. As another example, the machine-learned model(s) can process the statistical data to generate a diagnostic output.

In some implementations, the input to the machine-learned model(s) of the present disclosure can be sensor data. The machine-learned model(s) can process the sensor data to generate an output. As an example, the machine-learned model(s) can process the sensor data to generate a recognition output. As another example, the machine-learned model(s) can process the sensor data to generate a prediction output. As another example, the machine-learned model(s) can process the sensor data to generate a classification output. As another example, the machine-learned model(s) can process the sensor data to generate a segmentation output. As another example, the machine-learned model(s) can process the sensor data to generate a segmentation output. As another example, the machine-learned model(s) can process the sensor data to generate a visualization output. As another example, the machine-learned model(s) can process the sensor data to generate a diagnostic output.

In some cases, the input includes visual data, and the task is a computer vision task. In some cases, the input includes pixel data for one or more images, and the task is an image processing task. For example, the image processing task can be image classification, where the output is a set of scores, each score corresponding to a different object class and representing the likelihood that the one or more images depict an object belonging to the object class. The image processing task may be object detection, where the image processing output identifies one or more regions in the one or more images and, for each region, a likelihood that region depicts an object of interest. As another example, the image processing task can be image segmentation, where the image processing output defines, for each pixel in the one or more images, a respective likelihood for each category in a predetermined set of categories. As another example, the set of categories can be object classes.

FIG. 1A illustrates one example computing system that can be used to implement the present disclosure. Other computing systems can be used as well. For example, in some implementations, the user computing device 102 can include the model trainer 160 and the training dataset 162. In such implementations, the models 120 can be both trained and used locally at the user computing device 102. In some of such implementations, the user computing device 102 can implement the model trainer 160 to personalize the models 120 based on user-specific data.

FIG. 1B depicts a block diagram of an example computing device 10 that performs according to example embodiments of the present disclosure. The computing device can be a user computing device or a server computing device.

The computing device 10 includes a number of applications (e.g., applications 1 through N). Each application contains its own machine learning library and machine-learned model(s). For example, each application can include a machine-learned model. Example applications include a text messaging application, an email application, a dictation application, a virtual keyboard application, a browser application, etc.

As illustrated in FIG. 1B, each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

FIG. 1C depicts a block diagram of an example computing device 50 that performs according to example embodiments of the present disclosure. The computing device 50 can be a user computing device or a server computing device.

The computing device 50 includes a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer includes a number of machine-learned models. For example, as illustrated in FIG. 1C, a respective machine-learned model (e.g., a model) can be provided for each application and managed by the central intelligence layer. In other implementations, two or more applications can share a single machine-learned model. For example, in some implementations, the central intelligence layer can provide a single model (e.g., a single model) for all of the applications. In some implementations, the central intelligence layer is included within or otherwise implemented by an operating system of the computing device 50.

The central intelligence layer can communicate with a central device data layer. The central device data layer can be a centralized repository of data for the computing device 50. As illustrated in FIG. 1C, the central device data layer can communicate with a number of other components of the computing device, such as, for example, one or more sensors, a context manager, a device state component, and/or additional components. In some implementations, the central device data layer can communicate with each device component using an API (e.g., a private API).

### Example Model Arrangements

In some implementations, the systems and methods can include graph neural networks (GNN) and deep neural networks (DNN) for processing data. The systems and methods can factor in the normalized binding energy (NBE) and concentration of the molecules in the mixture to better understand the mixture and how the mixture may act. Graph neural networks (GNN), deep neural networks (DNN), and normalized binding energy (NBE) may be denoted as their respective acronyms, and concentration may be denoted such that the concentration of X is denoted as [X].

In some implementations, the system can include factoring in concentration dependence into the prediction followed by modeling the mixture as a whole. The system can include generating a molecule embedding by processing molecule data with a GNN to generate a molecule embedding (i.e., molecule_embedding=GNN(molecule)). The molecule embedding can then be processed with a DNN to generate NBE data (i.e., NBE=DNN(molecule_embedding)). The NBE of a molecule and the concentration of the molecule in the mixture may then be processed by various layers, which can include a softmax layer, and can be pooled with all other processed NBEs and concentrations of the other molecules in the mixture to generate receptor activation data (e.g., receptor_activations=sum(softmax([NBE+log [M], 0])[:−1])). In some implementations, the generated receptor activation data may then be processed with a DNN to generate perceptual odor response data (i.e., perceptual_odor_response=DNN(receptor_activations)). Alternatively and/or additionally, the system may simplify the process to include processing the molecule data with a GNN to generate a molecule embedding (i.e., molecule_embedding=GNN(molecule)), and the molecule embedding may then be processed with a DNN to generate perceptual odor response data (i.e., perceptual_odor_response=DNN(molecule_embedding)).

In some implementations, the systems and methods can determine a proper substrate score and/or generate feature vectors to aid in modeling mixtures and generating property predictions. In some implementations, a proper substrate score can be determined by processing a molecule embedding with a DNN, applying a sigmoid activation function, and concatenating the results (e.g., proper_substrate_score=concat(sigmoid(DNN(molecule_embedding)), [0])). Similarly, feature vectors may be generated using the concentration of the molecules, the normalized binding energy of the molecules, and a softmax activation function (e.g., OR_vector=softmax([NBE+log [M], 0])). In mixture modeling, the proper substrate score and the feature vectors may then be used to determine receptor activation data by scaling the vectors with the scores, then summing the results (e.g., receptor_activations=sum(proper_substrate_score*OR_vector)). Moreover, the receptor activation data can then be used for determining perceptual odor response data (e.g., perceptual_odor_response=DNN(receptor_activations)).

Inhibition of molecules can be factored into the predictions, in some implementations. For example, the systems and methods can determine inhibition data related to the normalized binding energy through a similar process to determining the normalized binding energy of a molecule. Molecule data can be processed by a GNN to generate a molecule embedding, and the molecule embedding can then be processed by a DNN to generate inhibition data, which can be denoted as inhibition_NBE=DNN(molecule_embedding). The inhibition data can then be used to determine receptor inhibition data by processing the inhibition data and concentration data of each molecule with various layers including a softmax layer and summing the results (e.g., receptor_inhibitions=sum(softmax([inhibition_NBE+log[M], 0])[:−1])). Receptor activation data and receptor inhibition data can be used to calculate net receptor activation data (e.g., net_receptor_activations=receptor_activations*(1−receptor_inhibitions)), which can be used to generate perceptual odor response data with a DNN (e.g., perceptual_odor_response =DNN(net_receptor_activations)).

In some implementations, each perceptual odor response function and models may be factored into the overall property predictions for the mixtures. For example, concentration dependence, mixtures with competitive inhibition, and mixtures with noncompetitive inhibition may be factored into the overall machine-learned prediction model using various functions, architectures, and models.

In some implementations, the systems and methods may include a specialized framework for processing the molecules individually to determine the individual properties of the molecules with the embedding model, or a first machine-learned model. These systems and methods may include or otherwise leverage machine-learned models (e.g., graph neural networks) in conjunction with molecule chemical structure data to predict one or more perceptual (e.g., olfactory, gustatory, tactile, etc.) properties of a molecule. In particular, the systems and methods can predict the olfactory properties (e.g., humanly-perceived odor expressed using labels such as “sweet,” “piney,” “pear,” “rotten,” etc.) of a single molecule based on the chemical structure of the molecule. Moreover, in some implementations, a machine-learned graph neural network can be trained and used to process a graph that graphically describes the chemical structure of a molecule to predict olfactory properties of the molecule. In particular, the graph neural network can operate directly upon the graph representation of the chemical structure of the molecule (e.g., perform convolutions within the graph space) to predict the olfactory properties of the molecule. As one example, the graph can include nodes that correspond to atoms and edges that correspond to chemical bonds between the atoms. Thus, the systems and methods of the present disclosure can provide prediction data that predicts the smell of previously unassessed molecules through the use of machine-learned models. The individual-molecule machine-learned models can be trained, for example, using training data that includes descriptions of molecules (e.g., structural descriptions of molecules, graph-based descriptions of chemical structures of molecules, etc.) that have been labeled (e.g., manually by an expert) with descriptions of olfactory properties (e.g., textual descriptions of odor categories such as “sweet,” “piney,” “pear,” “rotten,” etc.) that have been assessed for the molecules.

Thus, the first machine-learned model, or the embedding model, may use graph neural networks for quantitative structure-odor relationship (QSOR) modeling. Learned embeddings from graph neural networks capture a meaningful odor space representation of the underlying relationship between structure and odor.

More particularly, the relationship between a molecule's structure and its olfactory perceptual properties (e.g., the scent of a molecule as observed by a human) is complex, and, to date, generally little is known about such relationships. Accordingly, the systems and methods of the present disclosure provide for the use of deep learning and under-utilized data sources to obtain predictions of olfactory perceptual properties of unseen molecules, thus allowing for improvements in the identification and development of molecules having desired perceptual properties, for example, allowing for development of new compounds useful in commercial flavor, fragrance, or cosmetics products, improving expertise in prediction of drug psychoactive effects from single molecules, and/or the like.

More particularly, according to one aspect of the present disclosure, machine-learned models, such as graph neural network models, can be trained to provide predictions of perceptual properties (e.g., olfactory properties, gustatory properties, tactile properties, etc.) of a molecule based on an input graph of the chemical structure of the molecule. For instance, a machine-learned model may be provided with an input graph structure of a molecule's chemical structure, for example, based on a standardized description of a molecule's chemical structure (e.g., a simplified molecular-input line-entry system (SMILES) string, etc.). The machine-learned model may provide output comprising a description of predicted perceptual properties of the molecule, such as, for example, a list of olfactory perceptual properties descriptive of what the molecule would smell like to a human. For instance, a SMILES string can be provided, such as the SMILES string “O═C(OCCC(C)C)C” for the chemical structure of isoamyl acetate, and the machine-learned model can provide as output a description of what that molecule would smell like to a human, for example, a description of the molecule's odor properties such as “fruit, banana, apple.” In particular, in some implementations, in response to receipt of a SMILES string or other description of chemical structure, the systems and methods can convert the string to a graph structure that graphically describes the two-dimensional structure of a molecule and can provide the graph structure to a machine-learned model (e.g., a trained graph convolutional neural network and/or other type of machine-learned model) that can predict, from either the graph structure or features derived from the graph structure, olfactory properties of the molecule. Additionally or alternatively to the two-dimensional graph, systems and methods could provide for creating a three-dimensional graph representation of the molecule, for example using quantum chemical calculations, for input to a machine-learned model.

In some examples, the prediction can indicate whether or not the molecule has a particular desired olfactory perceptual quality (e.g., a target scent perception, etc.). In some embodiments, the prediction data can include one or more types of information associated with a predicted olfactory property of a molecule. For instance, prediction data for a molecule can provide for classifying the molecule into one olfactory property class and/or into multiple olfactory property classes. In some instances, the classes can include human-provided (e.g., experts) textual labels (e.g., sour, cherry, piney, etc.). In some instances, the classes can include non-textual representations of scent/odor, such as a location on a scent continuum or the like. In some instances, prediction data for molecules can include intensity values that describe the intensity of the predicted scent/odor. In some instances, prediction data can include confidence values associated with the predicted olfactory perceptual property.

In addition or alternatively to specific classifications for a molecule, prediction data can include a numerical embedding that allows for similarity search, clustering, or other comparisons between two or more molecules based on a measure of distance between two or more embeddings. For example, in some implementations, the machine-learned model can be trained to output embeddings that can be used to measure similarity by training the machine-learned model using a triplet training scheme where the model is trained to output embeddings that are closer in the embedding space for a pair of similar chemical structures (e.g., an anchor example and a positive example) and to output embeddings that are more distant in the embedding space for a pair of dissimilar chemical structures (e.g., the anchor and a negative example). Moreover, the outputs of these models may be configured to be processed by a second machine-learned model for predicting the properties of a mixture of various models.

Thus, in some implementations, the systems and methods of the present disclosure may not necessitate the generation of feature vectors descriptive of the molecule for input to a machine-learned model. Rather, the machine-learned model can be provided directly with the input of a graph-value form of the original chemical structure, thus reducing the resources required to make olfactory property predictions. For example, by providing for the use of the graph structure of molecules as input to the machine-learned model, new molecule structures can be conceptualized and evaluated without requiring the experimental production of such molecule structures to determine perceptual properties, thereby greatly accelerating the ability to evaluate new molecular structures and saving significant resources.

Moreover, in some implementations, training data including a plurality of known molecules can be obtained to provide for training one or more machine-learned models (e.g., a graph convolutional neural network, other type of machine-learned model) to provide predictions of olfactory properties of molecules. For example, in some embodiments, the machine-learned models can be trained using one or more datasets of molecules, where the dataset can include the chemical structure and a textual description of the perceptual properties (e.g., descriptions of the smell of the molecule provided by human experts, etc.) for each molecule. As one example, the training data can be derived from industry lists such as, for example, publicly available perfume industry lists of chemical structures and their corresponding odors. In some embodiments, due to the fact that some perceptual properties are rare, steps can be taken to balance out common perceptual properties and rare perceptual properties when training the machine-learned model(s).

According to another aspect of the present disclosure, in some embodiments, the systems and methods may provide for indications of how changes to a molecule structure could affect the predicted perceptual properties. These changes may be later processed by the second machine-learned model to generate an interaction property prediction, which can be used to generate an overall mixture property prediction. For example, the systems and methods could provide indications of how changes to the molecule structure may affect the intensity of a particular perceptual property, how catastrophic a change in the molecule's structure would be to desired perceptual qualities, and/or the like. In some implementations, the systems and methods may provide for adding and/or removing one or more atoms and/or groups of atoms from a molecule's structure to determine the effect of such addition/removal on one or more desired perceptual properties. For example, iterative and different changes to the chemical structure can be performed and then the result can be evaluated to understand how such change would affect the perceptual properties of the molecule. As yet another example, a gradient of the classification function of the machine-learned model can be evaluated (e.g., with respect to a particular label) at each node and/or edge of the input graph (e.g., via backpropagation through the machine-learned model) to generate a sensitivity map (e.g., that indicates how important each node and/or edge of the input graph was for output of such particular label). Further, in some implementations, a graph of interest can be obtained, similar graphs can be sampled by adding noise to the graph, and then the average of the resulting sensitivity maps for each sampled graph can be taken as the sensitivity map for the graph of interest. Similar techniques can be performed to determine perceptual differences between different molecule structures.

In some implementations, the systems and methods can provide for interpreting and/or visualizing which aspects of a molecule's structure most contributes to its predicted odor quality. For example, in some implementations, a heat map can be generated to overlay the molecule structure that provides indications of which portions of a molecule's structure are most important to the perceptual properties of the molecule and/or which portions of a molecule's structure are less important to the perceptual properties of the molecule. In some implementations, data indicative of how changes to a molecule structure would impact olfactory perception can be used to generate visualizations of how the structure contributes to a predicted olfactory quality. For example, as described above, iterative changes to the molecule's structure (e.g., a knock-down technique, etc.) and their corresponding outcomes can be used to evaluate which portions of the chemical structure are most contributory to the olfactory perception. As another example, as described above, a gradient technique can be used to generate a sensitivity map for the chemical structure, which can then be used to produce the visualization (e.g., in the form of a heat map).

Furthermore, in some implementations, machine-learned model(s) may be trained to produce predictions of a molecule chemical structure that would provide one or more desired perceptual properties (e.g., generate a molecule chemical structure that would produce a particular scent quality, etc.). For example, in some implementations, an iterative search can be performed to identify proposed molecule(s) that are predicted to exhibit one or more desired perceptual properties (e.g., targeted scent quality, intensity, etc.). For instance, an iterative search can propose a number of candidate molecule chemical structures that can be evaluated by the machine-learned model(s). In one example, candidate molecule structures can be generated through an evolutionary or genetic process. As another example, candidate molecule structures can be generated by a reinforcement learning agent (e.g., recurrent neural network) that seeks to learn a policy that maximizes a reward that is a function of whether the generated candidate molecule structures exhibit the one or more desired perceptual properties.

Thus, in some implementations, a plurality of candidate molecule graph structures that describe the chemical structure of each candidate molecule can be generated (e.g., iteratively generated) for use as input to a machine-learned model. The graph structure for each candidate molecule can be input to the machine-learned model to be evaluated. The machine-learned model can produce prediction data for each candidate molecule or a group of molecules that describes one or more perceptual properties of the one or more candidate molecules. The candidate molecule prediction data can then be compared to the one or more desired perceptual properties to determine if the candidate molecule(s) would exhibit desired perceptual properties (e.g., a viable molecule candidate, etc.). For example, the comparison can be performed to generate a reward (e.g., in a reinforcement learning scheme) or to determine whether to retain or discard the candidate molecule (e.g., in an evolutionary learning scheme). Brute force search approaches may also be employed. In further implementations, which may or may not have the evolutionary or reinforcement learning structures described above, the search for candidate molecules that exhibit the one or more desired perceptual properties can be structured as a multi-parameter optimization problem with a constraint on the optimization defined for each desired property.

The systems and methods may provide for predicting, identifying, and/or optimizing other properties associated with a molecule structure along with desired olfactory properties. For example, the machine-learned model(s) may predict or identify properties of molecule structures such as optical properties (e.g., clarity, reflectiveness, color, etc.), gustatory properties (e.g., tastes like “banana,” “sour,” “spicy,” etc.) shelf-stability, stability at particular pH levels, biodegradability, toxicity, industrial applicability, and/or the like.

According to another aspect of the present disclosure, the machine-learned models described herein can be used in active learning techniques to narrow a wide field of candidates to a smaller set of molecules or mixtures that are then manually evaluated. According to other aspects of the present disclosure, systems and methods can allow for synthesis of molecules, and/or mixtures, with particular properties in an iterative design-test-refine process. For example, based on prediction data from the machine-learned models, molecules or mixtures can be proposed for development. The molecules or mixtures can then be synthesized, and then can be subjected to specialized testing. Feedback from the testing can then be provided back to the design phase to refine the molecules to better achieve desired properties, etc.

Methods, architectures, motivations, and practices utilized in molecule property prediction can be employed or utilized in the other initial predictions and may be utilized in the overall mixture property predictions.

In some implementations, some property predictions may be determined based on a first determined property prediction. The secondary determined property predictions can be determined by utilizing known transfer properties and a non-learned general purpose descriptor (e.g., SMILES string, Morgan fingerprint, Dragon descriptor, etc.). These descriptors are generally intended to “featurize” a molecule, rather than convey complicated structural interrelations. For instance, some existing approaches featurize or represent the molecule with general purpose heuristic features, such as Morgan fingerprints or Dragon descriptors. However, the general purpose featurization strategies often do not highlight the important information related to specific tasks, such as predicting the olfactory or other sensory properties of molecules in a given species. For instance, Morgan fingerprints are generally designed for “lookup” of similar molecules. Morgan fingerprints generally do not include spatial arrangement of a molecule. While this information can nonetheless be useful, it may be insufficient alone, in some design cases, such as olfactory cases which may benefit from spatial understanding. Despite this, a scratch-trained model with a low amount of available training data is unlikely to beat a Morgan fingerprint model.

Another existing approach is physics-based modeling of sensory properties. For instance, physics-based modeling can include computational modeling of sensory (e.g., olfactory) receptors or sensory-related (e.g., olfactory-related) proteins. For instance, given a computational model of the olfactory receptor target, it is possible to run high throughput docking screens to find molecular candidates for desired tasks. However, this can be complicated for certain tasks, as it can be computationally expensive to model all possible interactions for all candidates. Furthermore, physics-based modeling of sensory performance can require explicit knowledge about the task at hand, such as the physical structure of a receptor, its binding pocket, and the positioning of a chemical ligand in that pocket, which may not be readily available. Furthermore, while some properties (e.g., pharmaceutical properties, material properties) of a molecule may be easily learned, some sensory/perception properties in particular, such as sensory properties (e.g., olfactory properties), can be challenging to make predictions for. This can be further complicated by the fact that a base, such as ethanol, plastic, shampoo, soap, fabric, etc., for certain scented chemicals can affect the perceived smell of the chemical. For instance, the same chemical may be perceived differently in an ethanol base compared to, for example, a soap base. Thus, even for chemicals that have a large amount of available training data in one base, there may be a limited amount of data in another base.

For example, in the domain of insect repellents, some potential repellants may act either as antagonists or as secondary inhibitors, and it would be computationally expensive to model each possible interaction. In addition, the physical structure of only many sensory receptors may be unavailable, which lends that traditional docking simulation may be impossible. For instance, from the insect repellent screening perspective, existing methods used to predict chemical properties involve simulating the docking of a specific molecule in a receptor pocket via detailed molecular dynamics simulation or binding mode prediction. However, these methods require expensive or difficult-to-acquire prior data in order to function in a new domain, such as the crystal structure of a specific receptor to be bound. Since perception (e.g., scent, taste) is the result of the collaborative activation of many hundreds of receptor types, and the crystal structure of very few receptors involved in chemical perception are known, this approach is often not possible or overly complicated.

Example aspects of the present disclosure can provide solutions for these and other challenges. According to an aspect of the present disclosure, a machine-learned sensory prediction model may be trained on a first sensory prediction task and used to output predictions associated with a second sensory prediction task. As one example, the first sensory prediction task may be a broader sensory prediction task than the second sensory prediction task. For example, the model may be trained on a broad task and transferred to a narrow task. As one example, the first task may be a broad property task, and the second task may be a specific property task (e.g., olfactory). Additionally and/or alternatively, the first sensory prediction task may be a task for which a larger amount of training data is available than for the second sensory prediction task. Additionally and/or alternatively, the first sensory prediction task may be associated with a first species and the second sensory prediction task may be associated with a second species. As one example, the first sensory prediction task may be a human olfactory task. Additionally and/or alternatively, the second sensory prediction task may be a pest control task, such as a mosquito repellent task.

As one example, a sensory embedding model can be trained to produce a sensory embedding for the first sensory prediction task. The sensory embedding can be learned from the first sensory prediction task, such as from a larger available dataset, such that the sensory embedding is specific to the first prediction task (e.g., a broader task). Despite being trained with regard to the first prediction task, however, it is recognized according to example aspects of the present disclosure that this sensory embedding can capture useful information for other (e.g., narrower) sensory prediction tasks. Furthermore, this sensory embedding can be transferred, fine-tuned, or otherwise modified to produce accurate predictions in another domain for the second sensory prediction task that has less available data then the first sensory prediction task, such as a task where machine learning or accurate prediction would otherwise be difficult and/or impossible.

As one example, a sensory embedding model can be trained in tandem with a first prediction task model. The sensory embedding model and the first prediction task model can be trained using (e.g., labeled) first prediction task training data for the first prediction task. For instance, the sensory embedding model can be trained to produce sensory embeddings with respect to the first prediction task. These sensory embeddings can capture information that is useful in the second prediction task. After training the sensory embedding model with the first prediction task model on first prediction task training data, the sensory embedding model can be used with a second prediction task model to output predictions associated with the second prediction task. In some cases, the sensory embedding model can further be refined, fine-tuned, or otherwise continually trained on second prediction task training data associated with the second prediction task. In some implementations, the model may be trained at a lower training rate with the second prediction task than for the first prediction task, to prevent intuitively un-learning the information learned from the first prediction task. In some implementations, an amount of second prediction task training data may be less than an amount of first prediction task training data, such as if there is less available data for the second prediction task than for the first prediction task.

The machine-learned models can be trained, for example, using training data that includes descriptions of molecules and/or mixtures (e.g., structural descriptions of molecules, graph-based descriptions of chemical structures of molecules, etc.) for a first sensory prediction task, such as molecules that have been labeled (e.g., manually by an expert) with descriptions of sensory properties (e.g., olfactory properties) (e.g., textual descriptions of odor categories such as “sweet,” “piney,” “pear,” “rotten,” etc.) that have been assessed for the molecules. For instance, these descriptions of olfactory molecules may relate to, for example, human perception. These models can then be used for a second sensory prediction task that is different from the first sensory prediction task. For instance, the second sensory prediction task may relate to non-human perception. For instance, in some implementations, the model is transferred across different species' perceptual properties of molecules.

In this way, a model that is trained on a large dataset can be transferred to a task having a smaller dataset while still achieving high predictive performance. In particular, it is observed that the sensory embeddings can provide a significant boost to prediction quality when transfer learning across species for sensory (e.g., olfactory) prediction tasks. Beyond even in-domain transfer learning, these sensory embeddings can provide improved performance for even more disparate qualities, such as cross-species perception. This is especially unexpected in the chemical domain. For instance, the sensory embeddings may be taken directly as input at a second prediction task model. The sensory embedding model may then be fine-tuned and trained on the second sensory prediction task. Unexpectedly, the second sensory prediction task and the first sensory prediction task need not be overly similar. For instance, prediction tasks having sufficient distinction (e.g., cross-species, cross-domain, etc.) may nonetheless find benefit according to example aspects of the present disclosure.

Thus, some example aspects of the present disclosure are directed to propose the use of neural networks, such as graph neural networks, for olfactory, gustatory, and/or other sensory modeling across distinct domains, such as quantitative structure-odor relationship (QSOR) modeling. Graph neural networks can represent spatial information, which can be important for olfactory and/or other sensory modeling. Example implementations of the systems and methods described herein significantly outperform prior methods on a novel data set labeled by olfactory experts. Furthermore, the learned sensory embeddings from graph neural networks capture a meaningful odor space representation of the underlying relationship between structure and odor. These learned sensory embeddings can unexpectedly be applied to domains other than the domain for which the model used to generate the sensory embedding is learned. For example, a model trained on human sensory perception data may unexpectedly achieve desirable results outside of the human sensory perception domain, such as other species' perception and/or other domains. For instance, the use of graph neural networks can provide spatial understanding to the model that is beneficial for sensory modeling applications.

In some implementations, prediction for a first prediction task and/or the second prediction task can indicate whether or not the molecule has a particular desired sensory quality (e.g., a target scent perception, etc.). In some implementations, the prediction data can include one or more types of information associated with a predicted sensory property (e.g., olfactory property) of a molecule. For instance, prediction data for a molecule can provide for classifying the molecule into one sensory property (e.g., olfactory property) class and/or into multiple sensory property (e.g., olfactory property) classes. In some instances, the classes can include human-provided (e.g., experts) textual labels (e.g., sour, cherry, piney, etc.). In some instances, the classes can include non-textual representations of scent/odor, such as a location on a scent continuum or the like. In some instances, prediction data for molecules can include intensity values that describe the intensity of the predicted scent/odor. In some instances, prediction data can include confidence values associated with the predicted olfactory perceptual property. As another example, in some implementations, the prediction data may be descriptive of how well the molecule will perform at a particular task (e.g., a pest control task).

In addition or alternatively to specific classifications for a molecule, prediction data can include a numerical sensory embedding that allows for similarity search, clustering, or other comparisons between two or more molecules based on a measure of distance between two or more sensory embeddings. For example, in some implementations, the machine-learned model can be trained to output sensory embeddings that can be used to measure similarity by training the machine-learned model using a triplet training scheme where the model is trained to output sensory embeddings that are closer in the sensory embedding space for a pair of similar chemical structures (e.g., an anchor example and a positive example) and to output sensory embeddings that are more distant in the sensory embedding space for a pair of dissimilar chemical structures (e.g., the anchor and a negative example). According to example aspects of the present disclosure, these output sensory embeddings may be used even in dissimilar tasks such as cross-species tasks.

According to another aspect of the present disclosure, training data including a plurality of known molecules can be obtained to provide for training one or more machine-learned models (e.g., a graph convolutional neural network, other type of machine-learned model) to provide predictions of sensory properties (e.g., olfactory properties) of molecules. For example, in some embodiments, the machine-learned models can be trained using one or more datasets of molecules, where the dataset includes the chemical structure and a textual description of the perceptual properties (e.g., descriptions of the smell of the molecule provided by human experts, etc.) for each molecule. As one example, the training data can be derived from publicly available data such as, for example, publicly available lists of chemical structures and their corresponding odors. In some embodiments, due to the fact that some perceptual properties are rare, steps can be taken to balance out common perceptual properties and rare perceptual properties when training the machine-learned model(s). According to example aspects of the present disclosure, the training data may be provided for a first sensory prediction task, where the training data is more widely available than for a second sensory prediction task that is an overall objective of the model. The model may then be retrained for the second sensory prediction task on a (limited) amount of training data for the second sensory prediction task and/or used as-is for the second sensory prediction task without further training.

Moreover, in some implementations, the systems and methods may provide for indications of how changes to a molecule structure could affect the predicted perceptual properties (e.g., for the second prediction task). For example, the systems and methods could provide indications of how changes to the molecule structure may affect the intensity of a particular perceptual property, how catastrophic a change in the molecule's structure would be to desired perceptual qualities, and/or the like. In some embodiments, the systems and methods may provide for adding and/or removing one or more atoms and/or groups of atoms from a molecule's structure to determine the effect of such addition/removal on one or more desired perceptual properties. For example, iterative and different changes to the chemical structure can be performed and then the result can be evaluated to understand how such change would affect the perceptual properties of the molecule. As yet another example, a gradient of the classification function of the machine-learned model can be evaluated (e.g., with respect to a particular label) at each node and/or edge of the input graph (e.g., via backpropagation through the machine-learned model) to generate a sensitivity map (e.g., that indicates how important each node and/or edge of the input graph was for output of such particular label). Further, in some implementations, a graph of interest can be obtained, similar graphs can be sampled by adding noise to the graph, and then the average of the resulting sensitivity maps for each sampled graph can be taken as the sensitivity map for the graph of interest. Similar techniques can be performed to determine perceptual differences between different molecular structures.

Furthermore, the systems and methods of the present disclosure can provide for interpreting and/or visualizing which aspects of a molecule's structure most contributes to a predicted sensory quality (e.g., for the second prediction task). For example, in some embodiments, a heat map could be generated to overlay the molecule structure that provides indications of which portions of a molecule's structure are most important to the perceptual properties of the molecule and/or which portions of a molecule's structure are less important to the perceptual properties of the molecule. In some implementations, data indicative of how changes to a molecule structure would impact olfactory perception can be used to generate visualizations of how the structure contributes to a predicted olfactory quality. For example, as described above, iterative changes to the molecule's structure (e.g., a knock-down technique, etc.) and their corresponding outcomes can be used to evaluate which portions of the chemical structure are most contributory to the olfactory perception. As another example, as described above, a gradient technique can be used to generate a sensitivity map for the chemical structure, which can then be used to produce the visualization (e.g., in the form of a heat map).

The machine-learned model(s) may be trained to produce predictions of a molecule chemical structure or a mixture chemical formulation that would provide one or more desired perceptual properties (e.g., generate a molecule chemical structure that would produce a particular scent quality, etc.). For example, in some implementations, an iterative search can be performed to identify proposed molecule(s) or mixtures that are predicted to exhibit one or more desired perceptual properties (e.g., targeted scent quality, intensity, etc.). For instance, an iterative search can propose a number of candidate molecule chemical structures or mixture chemical formulations that can be evaluated by the machine-learned model(s). In one example, candidate molecule structures can be generated through an evolutionary or genetic process. As another example, candidate molecule structures can be generated by a reinforcement learning agent (e.g., recurrent neural network) that seeks to learn a policy that maximizes a reward that is a function of whether the generated candidate molecule structures exhibit the one or more desired perceptual properties. According to example aspects of the present disclosure, this perceptual property analysis can be related to a second sensory prediction task that is different from the first sensory prediction task.

The systems and methods may provide for predicting, identifying, and/or optimizing other properties associated with a molecule structure along with desired sensory properties (e.g., olfactory properties). For example, the machine-learned model(s) may predict or identify properties of molecule structures such as optical properties (e.g., clarity, reflectiveness, color, etc.), olfactory properties (e.g., scents such as scents reminiscent of scents of fruits, flowers, etc.), gustatory properties (e.g., tastes like “banana,” “sour,” “spicy,” etc.) shelf-stability, stability at particular pH levels, biodegradability, toxicity, industrial applicability, and/or the like for a second sensory prediction task that is different from a first sensory prediction task on which the model(s) were earlier trained.

In some implementations, the machine-learned models can be used in active learning techniques to narrow a wide field of candidates to a smaller set of molecules or mixtures that are then manually evaluated. Alternatively and/or additionally, the systems and methods can allow for synthesis of molecules or mixtures with particular properties in an iterative design-test-refine process. For example, based on prediction data from the machine-learned models, mixtures can be proposed for development. The mixtures can then be formulated, and then can be subjected to specialized testing. Feedback from the testing can then be provided back to the design phase to refine the mixtures to better achieve desired properties, etc. For example, results from the testing can be used as training data to re-train the machine-learned model. After re-training, predictions from the model can then again be used to identify certain molecules or mixtures for testing. Thus, an iterative pipeline can be evaluated where a model is used to select candidates and then testing results for the candidates can be used to re-train the model, and so on.

For instance, in one example implementation of the present disclosure, a model is trained using a large amount of human perceptual data, which may be readily available as training data. The model is then transferred to an at least somewhat related chemical problem, such as predicting whether a molecule or mixture will be a good mosquito repellent, discovering a new flavor molecule, etc. The model (e.g., a neural network) can also be packaged into a standalone molecule embedding tool for generating representations that focus on olfactory related problems. These representations can be used to search for odors that smell similarly or trigger similar behavior in animals. The embedding space described herein can additionally be useful as a codec for designing electronic scent perception systems (e.g., “electronic noses”).

As another example, certain sensory properties can be desirable for animal attractant and/or repellent tasks. For instance, the first sensory prediction task can be a human sensory task, such as human olfactory task, a human gustatory task, etc., based on chemical structure of a molecule or mixture. The first sensory property can be human perception properties, such as human olfactory perceptual properties and/or human gustatory perceptual properties. The second sensory prediction task can be a nonhuman sensory task, such as a related sensory task for another species. The second sensory prediction task can additionally and/or alternatively be or include performance of the molecule as an attractant and/or repellent for a certain species. For instance, the properties may indicate performance of the molecule at attracting a desired species (e.g., for incorporation into animal food, etc.), or repelling undesired species (e.g., an insect repellent).

For example, this can include pest control applications, such as mosquito repellent, insecticides, etc. For example, mosquito repellent may serve to repel mosquitoes and prevent bites contributing to transmission of viruses and diseases. For instance, services or technologies that relate to human and/or animal olfactory systems could potentially find use for systems and methods according to example aspects in various implementations. Example implementations can include, for example, approaches for finding suitable odors for insect repellent or other pest control, such as repellent for mosquitoes, pests that affect crop health, livestock health, personal health, building/infrastructure health, and/or other suitable pests. For instance, systems and methods described herein may be useful for designing a repellent, insecticide, attractant, etc. for a targeted species of insect or other animal, even animals for which little to no sensory perception data is available. As one example, the first sensory prediction task can be a sensory prediction task related to a human sense, such as a human olfactory task of predicting human olfactory perception labels based on molecular structure data. The second sensory prediction task may include predicting performance of molecules at repelling another species, such as mosquitos.

As another example, systems and methods according to example aspects of the present disclosure may find application in toxicology and/or other safety studies. For example, the first sensory prediction task and/or the second sensory prediction task may be toxicology prediction tasks. The sensory properties may relate to toxicity of chemicals based on chemical structures. As another example, systems and methods according to example aspects of the present disclosure can be beneficial in transferring to related olfactory tasks, such as discovering a molecule that will smell similar to an existing molecule, but with different physical properties such as color.

FIG. 2 depicts a block diagram of an example property prediction system 200 according to example embodiments of the present disclosure. In some implementations, the property prediction system 200 is trained to receive a set of input data 202, 204, 206, and 208 descriptive of molecules in a mixture and, as a result of receipt of the input data 202, 204, 206, and 208, provide output data 216 that includes one or more property predictions descriptive of predicted properties of a mixture. Thus, in some implementations, the property prediction system 200 can include one or more embedding model(s) 212 that are operable to generate molecule embeddings, and a machine-learned prediction model 214 that is operable to generate one or more property predictions 216.

Property prediction systems 200 can include two-stage processing of input data to generate one or more property predictions 216. For example, in the depicted system 200, the input data can include molecule data with respective molecule data 202, 204, 206, and 208 for each molecule in a mixture, in which the molecule data can be descriptive of an N number of molecules, and mixture data 210 descriptive of the composition of a mixture of the N number of molecules. The system 200 can process the molecule data with one or more embedding model(s) 212 to generate one or more embeddings to be processed by the machine-learned prediction model 214. In some implementations, the embedding model 212 can include a graph neural network (GNN) to generate one or more graphs. In some implementations, the molecule data can be processed such that the respective molecule data related to each individual molecule can be processed separately such that each embedding can represent a singular molecule.

The embeddings and the mixture data 210 can be processed by the machine-learned prediction model 214 to generate one or more property predictions 216. The machine-learned prediction model 214 can include a deep neural network and/or various other architectures. Moreover, the property predictions 216 can include various predictions related to various properties associated with the mixture. For example, the property predictions 216 may include sensory property predictions, such as an olfactory property prediction to later be used for creating a fragrance.

Furthermore, in this implementation, the first molecule 202, the second molecule 204, the third molecule 206, . . . , and the nth molecule 208 can be of the same or different concentrations in the theoreticized mixture. The system may weight the one or more embeddings based on concentration of the molecules. The weighting can be completed by the embedding model 212, the machine-learned prediction model 214, and/or a third separate weighting model.

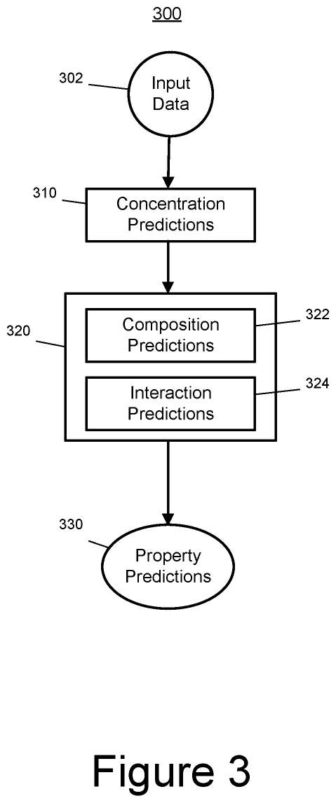

FIG. 3 depicts a block diagram of an example property prediction system 300 according to example embodiments of the present disclosure. The property prediction system 300 is similar to property prediction system 200 of FIG. 2 except that property prediction system 300 further includes three initial predictions.

More specifically, the depicted system 300 includes three initial predictions being made before the overall property predictions 330 are generated. For example, the system 300 can make individual molecule predictions 310, mixture composition property predictions 322, and mixture interaction property predictions 324, which can all be factored into the overall property predictions 330.

The system 300 can begin with obtaining input data 310, which can include molecule data and mixture data descriptive of a mixture with a set of molecules. The input data can be processed by a first model to generate molecule specific predictions 310, and in some implementations, the predictions 310 can be concentration specific predictions. The concentration predictions 310 may be weighted based on the concentration level. and the predictions of the various molecules may be pooled.

The output of the first model can then be processed by a second model 320, which can include two sub-models. The first sub-model can process the data and output composition specific property predictions 322 associated with the overall composition of the mixture. The second sub-model can process the data and output interaction specific property predictions 324 associated with predicted interactions in the mixture and/or predicted extrinsic interactions.

The three initial predictions can be processed to generate an overall property prediction 330 based on each of the initial predictions to allow for a better understanding of the mixture. For example, each individual molecule may have their own respective odor properties, while certain compositions may lead to some molecule properties being more prevalent. Moreover, interaction properties of various molecules and molecule sets may alter, enhance, or dilute certain odor properties. Therefore, each initial prediction can provide insight to how the overall mixture may smell, taste, etc.

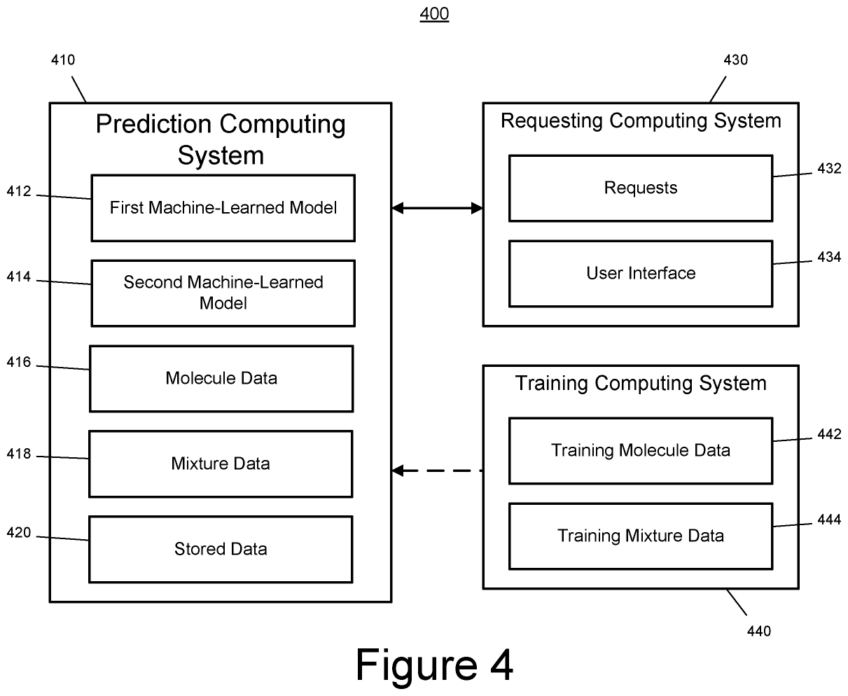

FIG. 4 depicts a block diagram of an example property prediction request system 400 according to example embodiments of the present disclosure. In some implementations, the property prediction request system 400 is trained to receive a set of training data 442 & 444 descriptive of known properties of individual molecules and known properties of mixture interactions and, as a result of receipt of the training data 442 & 444, determine and store property predictions for one or more mixtures. Thus, in some implementations, the property prediction request system 400 can include a prediction computing system 402 that is operable to predict and store mixture properties.

The property prediction request system 400 depicted in FIG. 4 includes a prediction computing system 410, a requesting computing system 430, and a training computing system 440 that can communicate with one another to make-up the overall system 400.

In some implementations, the property prediction request system can rely on a trained prediction computing system 410 that can predict and store properties of mixtures to later produce upon request. Training the prediction computing system 410 can include the use of a training computing system 440 that can provide training data for training the machine-learned models 412 & 414 of the prediction computing system 410. For example, the training computing system 440 may have training molecule data 442 for training a first machine-learned model (e.g., an embedding model) 412 and training mixture data 444 for training a second machine-learned model (e.g., a deep neural network model) 414. The training data can include known properties for various molecules, compositions, and interactions, and the training data, once received, may be stored in the prediction computing system for later reference. In some implementations, the training data can include labeled training datasets, which can include known properties of certain mixtures to complete ground truth training of the machine-learned models.

Moreover, the prediction computing system 410 may store molecule data 416 and mixture data 418 for reference, for retraining, or for centralization of data. Alternatively and/or additionally, the molecule data 416 may be sampled to generate a database of mixture property predictions. The sampling may be at random or may be influenced sampling based on known molecule properties, molecule categories, and/or molecule abundancy. The molecule data 416 and the mixture data 418 may be processed by the first machine-learned model 410 and the second machine-learned model to generate property predictions for mixtures to be stored 420 by the prediction system.

The stored data 420 may then be searchable or accessible via communication between the prediction computing system and the requesting computing system 430. The requesting computing system 430 can include a user interface 434 for a user to input a search query or a request related to a certain mixture or a certain property. In response to the input, the requesting computing system 430 can generate a request 432, which can be sent to the prediction computing system 410 to search or screen through the stored data to retrieve and provide one or more results. The one or more results can then be provided back to the requesting computing system, which may display the one or more results for the user via the user interface. In some implementations, the results may be one or more mixtures with a property prediction associated with or matching the search query/request. In some implementations, the results may be provided as mixture property profiles with the mixture and their respective property predictions.

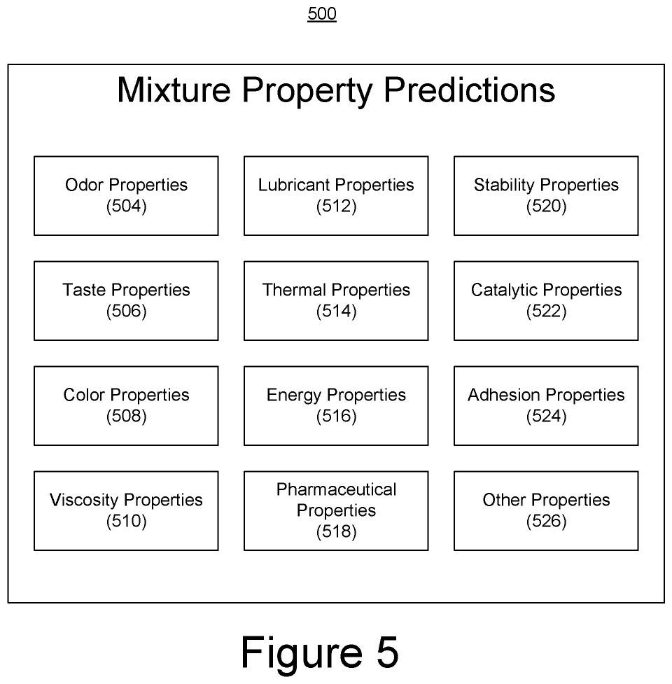

FIG. 5 depicts a block diagram of an example mixture property profile 500 according to example embodiments of the present disclosure. In some implementations, the mixture property profile 500 is trained to receive and store property predictions with their respective mixture for property screening or searching. Thus, in some implementations, the mixture property profile 500 can include various property predictions descriptive of predicted properties of a mixture.

The example mixture property profile 500 in FIG. 5 includes a grid of various property categories, which can be filled with property predictions, known properties, or a mix of known and predicted properties. In some implementations, the mixture property profiles 500 may include the mixture, the predicted properties, a graphical depiction of the mixture or molecules in the mixture, and/or reasons for the property predictions including initial predictions associated with the molecules in the mixture, the composition of the mixture, and/or the interactions in the mixture.

Some example properties displayed in a mixture property profile 500 can include odor properties 504, taste properties 506, color properties 508, viscosity properties 510, lubricant properties 512, thermal properties 514, energy properties 516, pharmaceutical properties 518, stability properties 520, catalytic properties 522, adhesion properties 524, and other miscellaneous properties 526,

Each property can be searchable for retrieving a mixture with a desired property upon request or query. Moreover, each property may provide a desired insight for use in a variety of different fields including consumer facing, industrial facing, etc. For example, odor properties 504 can include odor quality properties and odor intensity properties, which can be utilized in order to make fragrances, perfumes, candles, and so forth. Taste properties 506 can be utilized to make artificial flavors for candy, vitamins, or other consumables. The property predictions can be based at least in part on predicted receptor interactions and activations. Other properties can be used for product marketing, such as color properties 508, which can be used to predict the mixtures color or may include coloration properties. The coloration properties can be predicted to determine if the mixture could color other products. The viscosity properties 510 can be another property predicted and stored.

Other property predictions can be related to industrial applications such as providing lubricant properties 512 for machinery dynamics, and energy properties 516 can be used for producing better batteries. Pharmaceuticals may also be improved by or formulated based on knowledge obtained from these property predictions.

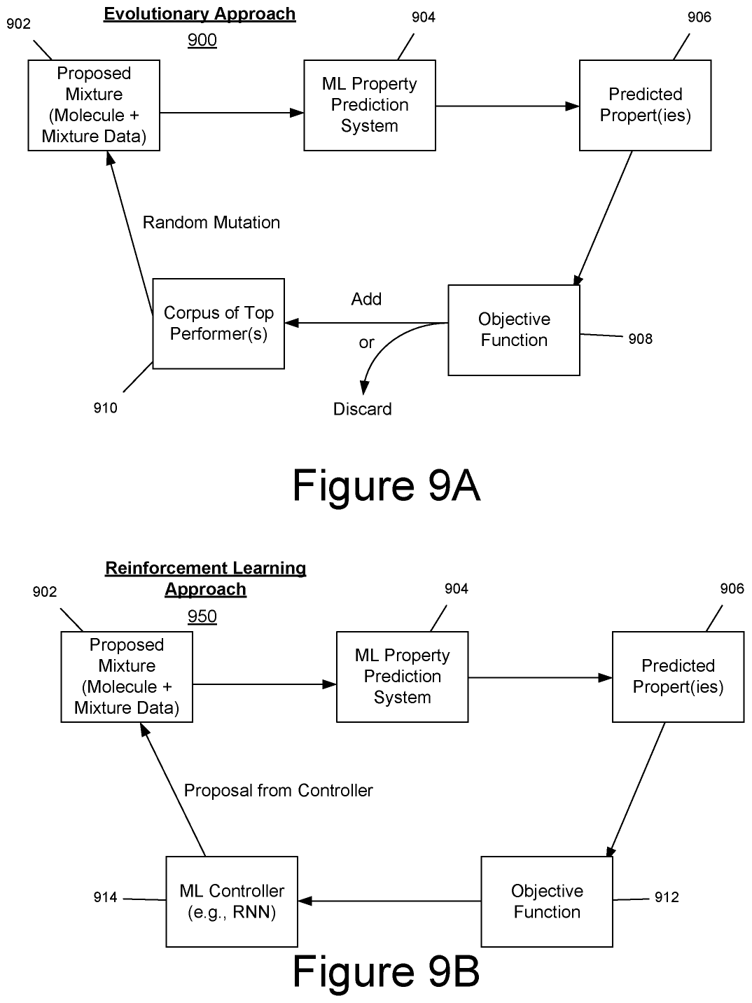

FIG. 9A depicts an example evolutionary approach 900, which can be used for generating a database of new mixtures with predicted properties. The proposed mixtures can have molecule data and mixture data 902 for each respective proposed mixture. The molecule data and mixture data 902 can be processed by the machine-learned property prediction system 904 to generate predicted properties 906 for the proposed mixture. The predicted properties 906 can then be processed by an objective function 908 to decide whether an addition to the corpus of top performers 910 should be made or whether to discard. A random mutation can be made, and the process can begin again. The evolutionary approach 900 can aid in generating a large database of useful mixtures to be available for screening by a human practitioner for use in a variety of products and industries.

FIG. 9B depicts an example reinforcement learning approach 950, which can be used for model optimization. Similar to the evolutionary approach 900, the reinforcement learning approach 950 can begin with molecule data and mixture data 902 of a proposed mixture being processed by a machine-learned property prediction system to generate predicted properties 906. The predicted properties 906 can then be processed by an objective function 912 to provide an output to a machine-learning controller 914 to provide a proposal to the system. In some implementations, the machine-learning controller can include a recurrent neural network. In some implementations, the reinforcement learning approach 950 can aid in refining the parameters of the machine-learned models disclosed herein.

### Example Methods

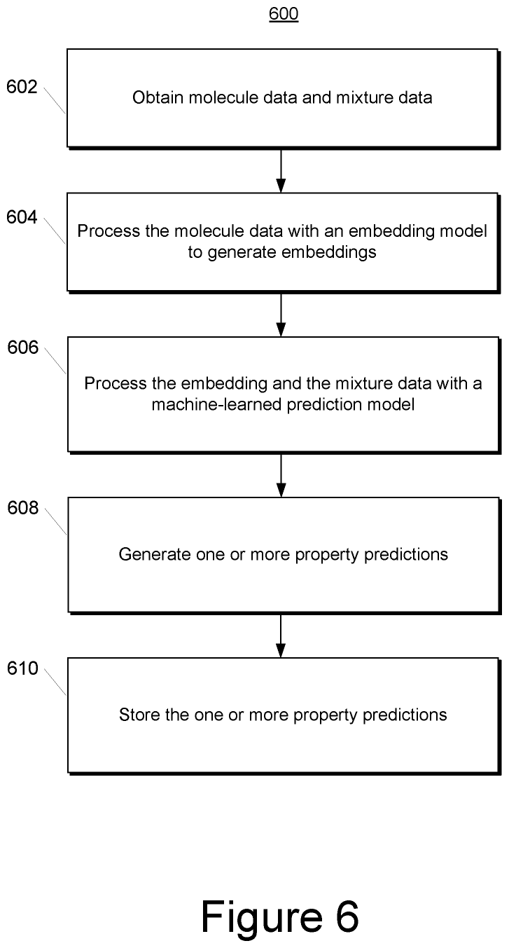

FIG. 6 depicts a flow chart diagram of an example method to perform according to example embodiments of the present disclosure. Although FIG. 6 depicts steps performed in a particular order for purposes of illustration and discussion, the methods of the present disclosure are not limited to the particularly illustrated order or arrangement. The various steps of the method 600 can be omitted, rearranged, combined, and/or adapted in various ways without deviating from the scope of the present disclosure.

At 602, a computing system can obtain molecule data and mixture data. The molecule data can be data descriptive of one or molecules of a mixture, and the mixture data can be descriptive of the mixture. In some implementations, the molecule data can include respective molecule data for each of a plurality of molecules, and the mixture data can describe the chemical formulation of the mixture. The data may be obtained via manually input data or automatically sampled data. In some implementations, the molecule data and the mixture data may be retrieved from a server. In some implementations, the mixture data can include concentrations for each of the molecules in the mixture.

At 604, the computing system can process the molecule data with an embedding model to generate one or more embeddings. The respective molecule data for each of the plurality of molecules can be processed with an embedding model to generate a respective embedding for each molecule. In some implementations, the embedding model can include a graph neural network to generate one or more graph embeddings. The embeddings can include embedded data descriptive of individual molecule properties.

At 606, the computing system can process the embeddings and the mixture data with a machine-learned prediction model. The machine-learned prediction model can include a deep neural network and may include a weighting model that can weight and pool the embeddings based on the respective molecule concentrations.

At 608, the computing system can generate one or more property predictions. The one or more property predictions can be based at least in part on the one or more embeddings and the mixture data. Moreover, the predictions can be based on individual molecule properties, concentration of molecules in the mixture, the composition of the mixture, and interaction properties of the mixture. In some implementations, the predictions can be sensory predictions, energy predictions, stability predictions, and/or thermal predictions.

At 610, the computing system can store the one or more property predictions. The property predictions may be stored in a searchable database for easy look-up of mixtures and properties.

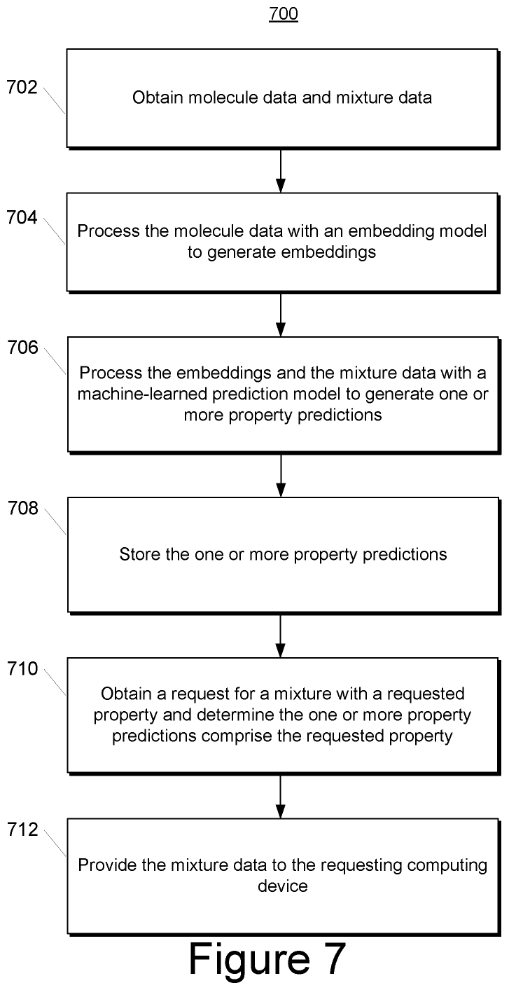

FIG. 7 depicts a flow chart diagram of an example method to perform according to example embodiments of the present disclosure. Although FIG. 7 depicts steps performed in a particular order for purposes of illustration and discussion, the methods of the present disclosure are not limited to the particularly illustrated order or arrangement. The various steps of the method 700 can be omitted, rearranged, combined, and/or adapted in various ways without deviating from the scope of the present disclosure.

At 702, a computing system can obtain molecule data and mixture data. In some implementations, the molecule data can be descriptive of a plurality of molecules in a mixture, and the mixture data can be descriptive of the mixture. The molecule data and mixture data may be obtained separately or at the same time.

At 704, the computing system can process the molecule data with an embedding model to generate embeddings. The embedding model can be a graph embedding model, in which the embeddings can be graph embeddings. In some implementations, the graph embeddings may be weighted and pooled to generate a graph of graphs. In some implementations, respective molecule data for each of the plurality of molecules can be processed as molecule specific sets with an embedding model to generate a respective embedding for each molecule.

At 706, the computing system can process the embeddings and the mixture data with a machine-learned prediction model to generate one or more property predictions. The property predictions can include predictions on a variety of mixture properties and can be used in a variety of fields and industries.

At 708, the computing system can store the one or more property predictions. The property predictions may be stored in a searchable database to provide easy access to the information.

At 710, the computing system can obtain a request for a mixture with a requested property and determine the one or more property predictions comprise the requested property. The request may be a formal request or may be a search query input into a user interface. In some implementations, the determination can include determining if a predicted property matches the requested property or is associated with the search query.

At 712, the computing system can provide the mixture data to the requesting computing device. The requesting computing device may receive the mixture data in a variety of forms including text data, graph data, etc. In some implementations, the mixture data may be provided with a mixture property profile that indicates the property predictions for the respective mixture.

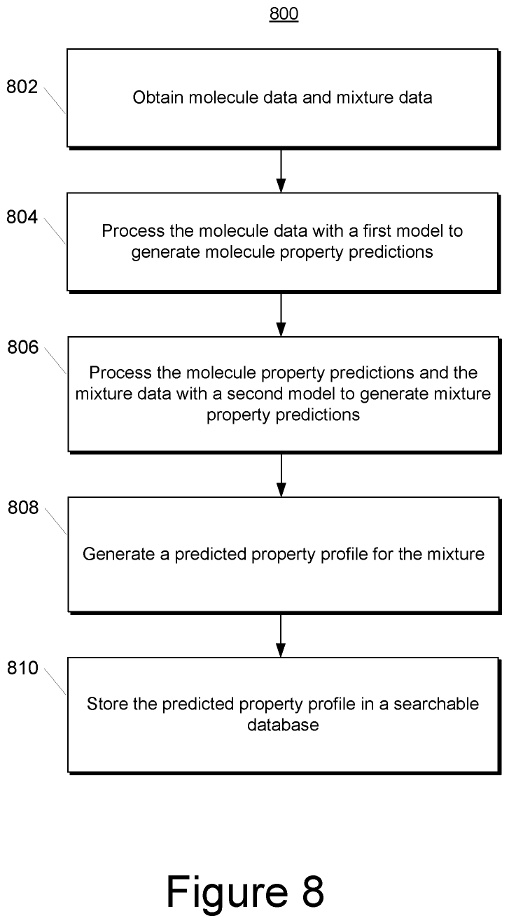

FIG. 8 depicts a flow chart diagram of an example method to perform according to example embodiments of the present disclosure. Although FIG. 8 depicts steps performed in a particular order for purposes of illustration and discussion, the methods of the present disclosure are not limited to the particularly illustrated order or arrangement. The various steps of the method 800 can be omitted, rearranged, combined, and/or adapted in various ways without deviating from the scope of the present disclosure.

At 802, a computing system can obtain molecule data and mixture data.

At 804, the computing system can process the molecule data with a first model to generate molecule property predictions. In some implementations, the molecule property predictions can be embedded before being processed by a second model.

At 806, the computing system can process the molecule property predictions and the mixture data with a second model to generate mixture property predictions. The mixture property predictions can be based at least in part on the molecule property predictions and concentrations of the one or more molecules.

At 808, the computing system can generate a predicted property profile for the mixture. The property profile can be organized data including the mixture, the mixture property predictions, and other data needed for application of the mixture in a desired field.

At 810, the computing system can store the predicted property profile in a searchable database. The searchable database can be enabled by other applications or may be a standalone searchable database with a dedicated interface.

### Additional Disclosure

The technology discussed herein makes reference to servers, databases, software applications, and other computer-based systems, as well as actions taken, and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

1. A computer-implemented method for mixture property prediction, the method comprising:
obtaining, by a computing system comprising one or more computing devices, respective molecule data for each of a plurality of molecules and mixture data associated with a mixture of the plurality of molecules;
respectively processing, by the computing device, the respective molecule data for each of the plurality of molecules with a machine-learned embedding model to generate a respective embedding for each molecule;
processing, by the computing system, the embeddings and the mixture data with a prediction model to generate one or more property predictions for the mixture of the plurality of molecules, wherein the one or more property predictions are based at least in part on the embeddings and the mixture data; and
storing, by the computing system, the one or more property predictions.

2. The method of claim 1, wherein the mixture data describes a respective concentration of each molecule in the mixture.

3. The method of claim 1, wherein the mixture data describes a composition of the mixture.

4. The method of claim 1, wherein the prediction model comprises a deep neural network.

5. The method of claim 1, wherein the machine-learned embedding model comprises a machine-learned graph neural network.

6. The method of claim 1, wherein the prediction model comprises a characteristic-specific model configured to generate predictions relative to a specific characteristic.

7. The method of claim 1, wherein the one or more property predictions are based at least in part on a binding energy of one or more molecules of the plurality of molecules.

8. The method of claim 1, wherein the one or more property predictions comprise one or more sensory property predictions.

9. The method of claim 1, wherein the one or more property predictions comprise an olfactory prediction.

10. The method of claim 1, wherein the one or more property predictions comprise a catalytic property prediction.

11. The method of claim 1, wherein the one or more property predictions comprise an energetic property prediction.

12. The method of claim 1, wherein the one or more property predictions comprise a surfactant between target property prediction.

13. The method of claim 1, wherein the one or more property predictions comprise a pharmaceutical property prediction.

14. The method of claim 1, wherein the one or more property predictions comprise a thermal property prediction.

15. The method of claim 1, wherein the prediction model comprises a weighting model configured to weight and pool the embeddings based on the mixture data, wherein the mixture data comprises concentration data related to the plurality of molecules of the mixture.

16. The method of claim 1, further comprising:
obtaining, by the computing system, a request from a requesting computing device for a chemical mixture with a requested property;
determining, by the computing system, the one or more property predictions satisfy the requested property; and
providing, by the computing system, the mixture data to the requesting computing device.

17. The method of claim 1, wherein the one or more property predictions are based at least in part on a molecule interaction property.

18. The method of claim 1, wherein the one or more property predictions are based at least in part on receptor activation data.

19. A computing system, the computing system comprising:
one or more processors;
one or more non-transitory computer readable media that collectively store instructions that, when executed by the one or more processors, cause the computing system to perform operations, the operations comprising:
obtaining respective molecule data for a plurality of molecules and mixture data associated with a mixture of the plurality of molecules, wherein the mixture data comprises concentrations for each respective molecule of the plurality of the molecules;
respectively processing the respective molecule data with an embedding model for each of the plurality of molecules to generate respective embeddings for each molecule;
processing the embeddings and the mixture data with a machine-learned prediction model to generate one or more property predictions, wherein the one or more property predictions are based at least in part on the embeddings and the mixture data; and
storing the one or more property predictions.

20. One or more non-transitory computer readable media that collectively store instructions that, when executed by one or more processors, cause a computing system to perform operations, the operations comprising:
obtaining respective molecule data for a plurality of molecules and mixture data associated with a mixture of the plurality of molecules;
respectively processing the respective molecule data with an embedding model for each of the plurality of molecules to generate respective embeddings for each molecule;
processing the embeddings and the mixture data with a machine-learned prediction model to generate one or more property predictions, wherein the one or more property predictions are based at least in part on the embeddings and the mixture data; and
storing the one or more property predictions.
